(19) United States
(12) Patent Application Publication
(10) Pub. No.: US 2010/0280247 A1
(43) Pub. Date: Nov. 4, 2010

(54) PROCESS IMPROVEMENT USING TMEDA

(75) Inventors: Stephan MUTTI, Paris (FR); Claude TOUM, Paris (FR); Patrick ROUSSEL, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: MILLENNIUM PHARMACEUTICALS INC., Cambridge, MA (US)

(21) Appl. No.: 12/786,951

(22) Filed: May 25, 2010

Related U.S. Application Data
(63) Continuation of application No. PCT/US2008/084610, filed on Nov. 25, 2008.
(60) Provisional application No. 60/991,277, filed on Nov. 30, 2007.

(51) Int. Cl. C07D 491/044

(52) U.S. Cl. 546/89

(57) ABSTRACT

An improved chemical synthesis for compounds having useful biological activity is disclosed, where the use of TMEDA or N,N,N′,N′-tetramethyl-ethane-1,2-diamine gives improved yield.

### FIELD OF THE INVENTION

This invention is directed to an improvement in synthetic processes for making chemical compounds having useful biological activity.

### BACKGROUND OF THE INVENTION

The present invention is an improvement in the synthetic preparation of 5-cyclopentyl-5-11-dihydro-10-oxa-1-aza-dibenzo[a,d]cyclohepten-5-ol, which is an intermediate used for the synthesis of biologically active compounds disclosed in U.S. Pat. No. 6,329,385.

### SUMMARY OF THE INVENTION

An improved chemical synthesis for compounds having useful biological activity is disclosed, where the use of TMEDA or N,N,N′,N′-tetramethyl-ethane-1,2-diamine gives improved yield. Both quality and yield have been significantly improved by adding TMEDA to cyclopropyl magnesium bromide. The chemical reaction between the Grignard reagent and the tricyclic ketone (1,2-addition to give a tertiary alcohol is limited by both enolisation of the ketone that decreases the rate of transformation and 1,4-addition, the main side reaction. Cyclopropyl magnesium bromide and TMEDA react to form a soluble complex. Thanks to the basicity of its two nitrogen atoms, TMEDA chelates magnesium, avoiding its chelation with the nitrogen atom the the tricyclic pyridine, hence the selectivity of the 1,2-addition is clearly better the rate of transformation of the ketone is improved as well.

### DETAILED DESCRIPTION OF THE INVENTION

In Scheme 1 is seen the reaction sequence used in the patent cited above.

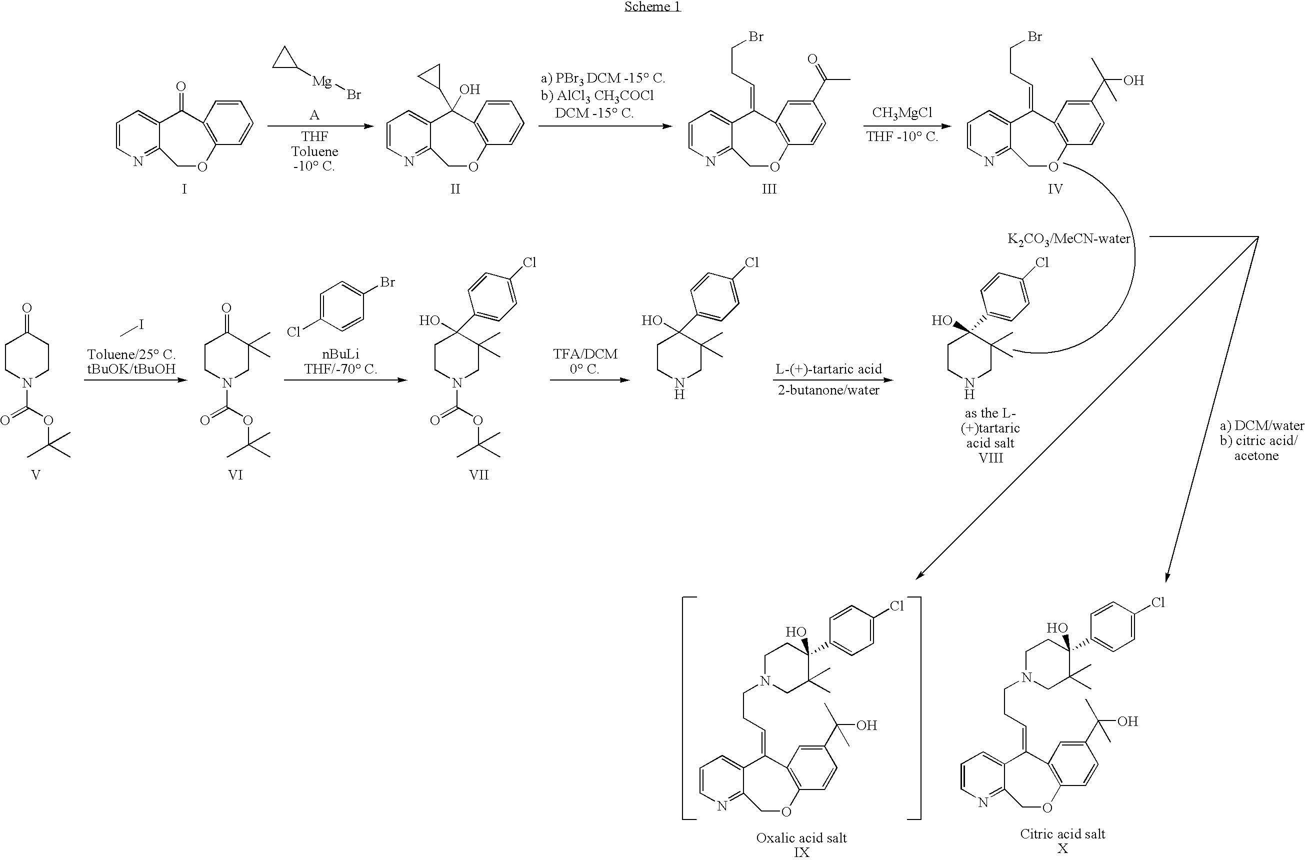

| Starting | | | |
|---|---|---|---|
| Material | | | Commercial Source or |
| Reference | | | Literature Reference for |
| Number | Name of Compound | Structure | the Preparation Method |
| I | 11H-10-oxa-1- azadibenzo[a,d] cyclohepten-5-one | 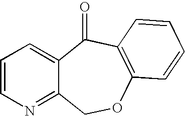 | Inoue, K.; Sugaya, T.; Ogasa, T. ; Tomioka, S. Synlett 1997, 113-116 |
| A | Cyclopropylmagnesium bromide (purchased as a 15.3% w/w solution in a mixture of THF and toluene) | 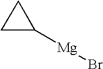 | Chemetall GMBH |
| A | Cyclopropylmagnesium bromide (1:1 complex with TMEDA and purchased as an 18.3% w/w solution in pure THF). | 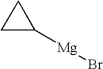 | Chemetall GMBH |

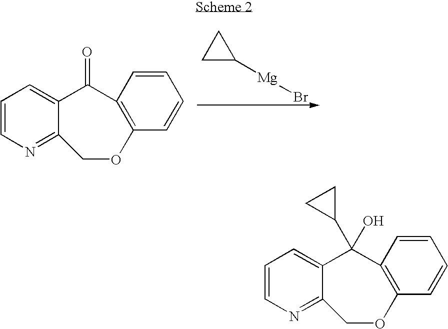

In Scheme 2 is seen the reaction being improved by the use of TMEDA disclosed here.

### EXAMPLES

### Example 1

### Synthesis of 5-cyclopropyl-5,11-dihydro-10-oxa-1-azadibenzo[a,d]cyclohepten-5-ol

### (Scheme 1, Compound II)

An 8 L jacketed dry glass reactor, equipped with an overhead stirrer, thermometer and a condenser, is purged with nitrogen. A solution of cyclopropyl magnesium bromide (2123 g, 2.23 moles, 15.3% w/w THF/toluene solution) and THF (1.78 L, anhydrous) is added and stirred. The resulting solution is cooled (−3° C.±5° C.) and the cyclopropyl magnesium bromide is precipitated partially. N,N,N′,N′-tetramethyl-ethane-1,2-diamine (TMEDA) (212 g, 1.80 mole) is charged over 1 hour and the reaction mixture is maintained below 0° C. to afford a clear solution. At −3° C.±5° C., a solution of 11H-10-oxa-1-azadibenzo[a,d]cyclohepten-5-one (250 g, 1.18 mole) in THF (750 mL, anhydrous) is added to the reaction mixture drop wise via a dropping funnel over 1 hour. The reaction mixture is stirred for 2 hours at −3±5° C. The progress of the reaction is monitored thereafter by HPLC. To quench, a solution of NH₄Cl (250 mL, aqueous saturated) is charged into the reaction mixture and stirred for 30 minutes. Acetic acid (348 g, diluted with 1.875 L of water) is charged while the temperature is raised to ˜20° C. and the reaction mixture is warmed to 50° C. The mixture is filtered over Clarcel® (175 g) and the filter cake is washed with THF (2×500 mL). The mother liquors and washes are mixed, allowed to separate and the aqueous layer is discarded. The organic layer is stirred and heated to remove THF (3.36 L) by distillation under atmospheric pressure. The final reactor temperature is 106° C. The resultant suspension is cooled (15° C./20° C.) and the off-white precipitate is filtered. The cake is washed with toluene (2×500 mL), water (2×500 mL), and is dried under vacuum (40 mmHg/50° C.) to yield final, desired 5-cyclopropyl-5,11-dihydro-10-oxa-1-azadibenzo[a,d]cyclohepten-5-ol (228.1 g, 76.3% yield). HPLC area=98%.

HPLC conditions: Column: INERTSIL® OD3 3 μm, 150×4.6mm; Column temperature: room temperature; Mobile phase: H₂O (600 mL): acetonitrile (400 mL): trifluoroacetic acid (0.2 mL); Flow rate: 1 mL/min; Pressure: 120 bars; Detection (UV): 220 nm; Injection volume: 20 μl; Analysis time: 35 min. R_{T} (11H-10-oxa-1-azadibenzo[a,d]cyclohepten-5-one)=11.2 min.; R_{T} (5 -cyclopropyl -5,11-dihydro-10-oxa-1-azadibenzo[a,d]cyclohepten-5 -ol)=3.4 min.; R_{T}=(4-cyclopropyl-4,11-dihydro-1H-10-oxa-1-azadibenzo[a,d]cyclohepten-5-one)=18.9 min.; and R_{T} (toluene)=28.0 min.

### Example 1a

### Synthesis of 5-cyclopropyl-5,11-dihydro-10-oxa-1-azadibenzo[a,d]cyclohepten-5-ol

### (Scheme 1, Compound II)

The title compound is prepared from 11H-10-oxa-1-azadibenzo[a,d]cyclohepten-5-one (7 kg) essentially as described above in Example 1. Isolated Yield=67%.

HPLC conditions: Column: INERTSIL OD3 3 μm, 150×4.6mm; Column temperature: room temperature; Mobile phase: H₂O (600 mL): acetonitrile (0.2 mL) : trifluoroacetic acid (0.2 mL); Flow rate: 1 mL/min; Pressure: 120 bars; Detection (UV): 220 nm; Injection volume: 20 μl; Analysis time: 35 min. R_{T} (11H-10-oxa-1-azadibenzo[a,d]cyclohepten-5-one)=11.2 min.; R_{T} (5-cyclopropyl-5,11-dihydro-10-oxa-1-azadibenzo[a,d]cyclohepten-5-ol)=3.4 min.; R_{T}=(4-cyclopropyl-4,11-dihydro-1H-10-oxa-1-azadibenzo[a,d]cyclohepten-5-one)=18.9 min.; and R_{T} (toluene)=28.0 min.

### Example 2

### Synthesis of 5-cyclopropyl-5,11-dihydro-10-oxa-1-azadibenzo[a,d]cyclohepten-5-ol

### (Scheme 1, Compound II)

A 2 L, 3-necked flask equipped with an overhead stirrer, thermometer and a condenser is purged with nitrogen. A solution of THF cyclopropylmagnesium bromide/TMEDA (355 g, 447 mmole of a 15.3% w/w) prepared from cyclopropylmagnesium bromide (as a 1:1 complex with TMEDA and purchased as an 18.3% w/w solution in THF from Chemetall Gmbh) and THF (360 mL, anhydrous), is added and stirred. The resulting solution is cooled (−5±5° C.). A solution of 11H-10-oxa-1-azadibenzo[a,d]cyclohepten-5-one (50 g, 236.7 mmole) in THF (150 mL, anhydrous) is added to the reaction solution dropwise via a dropping funnel over 1 hour. The reaction mixture is stirred for 1 hour at −5±5° C. The progress of the reaction is monitored thereafter by HPLC. To quench, a solution of NH₄Cl (50 mL, aqueous saturated) is charged into the reaction mixture and stirred for 30 minutes at 20° C. The reaction mixture is warmed to 45° C. Acetic acid (70 g, diluted with 375 mL of water) is charged over 10 minutes. The reaction mixture is stirred for an additional 25 minutes at 20° C. and is filtered over Clarcel® (35 g). The filter cake is washed with THF (2×50 mL). The mother liquors and washes are poured into a 2 L funnel and the aqueous layer is discarded. Into a 2 L, 3-necked flask equipped with an overhead stirrer, thermometer and a condenser are poured the organic layer and toluene (250 mL). THF (1050 mL) is removed by distillation under atmospheric pressure. The final reactor temperature is 100° C. to afford a suspension, which is cooled to 20° C. The white precipitate is stirred for 1 hour at 20° C., filtered, washed with toluene (2×100 mL) and water (2×100 mL), and is dried under vacuum (40 mmHg/50° C.) to afford desired 5-cyclopropyl-5,11-dihydro-10-oxa-1-azadibenzo[a,d]cyclohepten-5-ol (51 g, 85% yield). mp 210-212° C. HPLC area=99.5%.

HPLC conditions: Column: INERTSIL® OD3 3 μm, 150×4.6mm; Column temperature: room temperature; Mobile phase: H₂O (600 mL): acetonitrile (400 mL): trifluoroacetic acid (0.2 mL); Flow rate: 1 mL/minute; Pressure: 120 bars; Detection (UV): 220 nm; Injection volume: 20 μl; Analysis time: 35 min. R_{T} (11H-10-oxa-1-azadibenzo[a,d]cyclohepten-5-one)=11.2 min.; R_{T} (5-cyclopropyl-5,11-dihydro-10-oxa-1-azadibenzo[a,d]cyclohepten-5-ol)=3.4 min.; R_{T}=(4-cyclopropyl-4,11-dihydro-1H-10-oxa-1-azadibenzo[a,d]cyclohepten-5-one)=18.9 min.; and R_{T} (toluene)=28.0 min.

MS m/z (EI): 253 (M^{+.}), 225 (M-CO^{+.}), 212 (M-C₃H₅^{+.}, 184 (212-CO^{+.}). IR (KBr): 3393, 3084, 3066, 3007, 2960, 2882, 1582, 1487, 1449, 1441, 1425, 1282, 1216, 1052, 1042, 881, 806, 770, 744, 728 and 650 cm⁻¹.

H¹NMR (300 MHz, (CD₃)₂SO-d6, δin ppm): 0.24 (m, 2H), 0.49 (m, 1H), 0.59 (m, 1H), 1.99 (m, 1H), 5.00 (d, J=16 Hz, 1H), 5.47 (d, J=16 Hz, 1H), 5.75 (s, 1H), from 7.10 to 7.25 (m, 2H), from 7.25 to 7.40 (m, 2H), 7.63 (dd, J=7.5 and 1.5 Hz, 1H), 8.16 (d, J=8 and 1 Hz, 1H), 8.42 (dd, J=4.5 and 1 Hz, 1H).

The present invention may be embodied in other specific forms without departing from the spirit or essential attributes thereof.

1. An improved synthesis of 5-cyclopentyl-5-11-dihydro-10-oxa-1-aza-dibenzo[a,d]cyclohepten-5-ol, comprising:
carrying out the reaction: 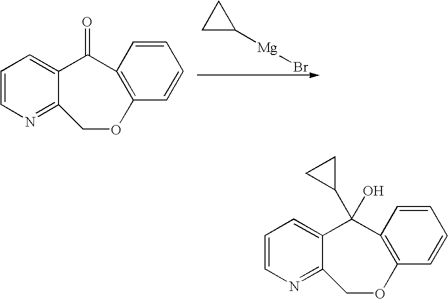
with TMEDA added.
